(19) United States
(12) Patent Application Publication
(10) Pub. No.: US 2016/0074773 A1
(43) Pub. Date: Mar. 17, 2016

(54) METHOD FOR SEPARATING AND PURIFING FUNCTIONAL INGREDIENTS FROM PLACENTA USING SUPERCRITICAL FLUID TECHNOLOGY

(71) Applicants: Super Well Biotechnology Corporation, Taichung City (TW); Ultra-Microrigin Biomedical Technology Co., Ltd., Hsinchu County (TW)

(72) Inventors: Zer-Ran YU, Taichung City (TW); Be-Jen WANG, Taichung City (TW); Shu-Mei LIN, Taichung City (TW); Hua-Ching LIN, Taichung City (TW); Ming-Hsi CHUANG, Hsinchu County (TW); Chu-Ting LIU, Hsinchu County (TW); I-Lung YU, Hsinchu County (TW); Chiu-Ying PENG, Hsinchu County (TW); Lin-Hsiang CHUANG, Hsinchu County (TW)

(21) Appl. No.: 14/702,388

(22) Filed: May 1, 2015

(30) Foreign Application Priority Data
Sep. 16, 2014 (TW) 103131963

(51) Int. Cl. B01D 11/02; A61K 38/17; A61K 38/18; A61K 35/50

(52) U.S. Cl. CPC B01D 11/0203; A61K 35/50; A61K 38/1709; A61K 38/18

(57) ABSTRACT

The present invention provides a method to separate and purify functional ingredients in placenta using supercritical fluid technology, where, placenta extract liquid and supercritical CO₂ solvent are guided into a fractionation tank constantly in a preset velocity at a preset temperature and pressure to extract peptides, proteins and other functional ingredients. And then, the solution is conveyed through the three fractionation tanks at a preset temperature and depressurized gradually to separate, fractionate and purify the content of placenta peptide and protein, so as to obtain the high purity of peptides, proteins, active factors and other functional ingredients.

The current application claims a foreign priority to application number 103131963 filed on Sep. 16, 2014 in Taiwan.

### BACKGROUND OF INVENTION

1. Field of the Invention

The present invention relates generally to a technology of separating active ingredients from placenta, and more particularly to a method which separates and purifies the functional ingredients from the placenta using supercritical fluid technology.

2. Description of Related Art

Placenta is a binding structure between the fetus and parent body and supplies all nutrients and protections for embryo. Scientific researchers have found that placenta contains abundant growth factors, hormones, proteins, polypeptides, nucleic acid and other condensed nutrients good for body health. It contains abundant growth factors such as hepatocyte growth factor (HGF), neurotrophic growth factor (NGF), epidermal growth factor (EGF), insulin-like growth factor (IGF), platelet-derived growth factor (PDGF), hematopoietic cell growth factors (HCGF), vascular epidermal growth factor (VEGF) and other growth factors to promote the growth of stem cells. These growth factors can regulate autonomic nervous system, improve cardiovascular circulation and wound healing, enhance the nerve regeneration, balance multiple hormones, reinforce immunity, and improve intestinal health.

There are many methods to extract bioactive peptides from the placenta, such as U.S. Pat. No. 3,409,605 adsorbs, concentrates and purifies growth factors in placenta through ion exchange resin; U.S. Pat. No. 4,169,139 separate proteins which are precipitable by acetone, dilute lead acetate and dilute ammonium sulfate solutions; China patents CN1298736C and CN102488713B wash the fresh placenta by sterile water. 10-100 times of saline solution are added and then homogenized with placenta tissue by high-speed homogenizer. The solution is desalinated through dialysis and then clarified by ultrafiltration membrane to obtain a sort of colorless or yellowish clarified liquid, which is then separated, purified through anion exchange chromatography, gel exclusion chromatography, reverse-phase high performance liquid chromatography, and lyophilization to obtain the purified product. China patent CN101658534A applies low temperature of liquid nitrogen method to extract active peptide ingredients based on high-speed pulverizing the placenta, dialyzed desalination, ultrafiltration membrane clarification, and under the condition of adsorption and elution through ion exchange resin.

Existing methods for extracting and purifying relevant functional ingredients are complicated, and those for separating and purifying peptides, protein and active factors are time-consuming and require a large amount of organic solvents, however, the organic solvents will cause chemical reaction with the functional ingredients during preparation course. Accordingly, not only functional ingredients will be destroyed in solvent heating and evaporation during the solvent removal and condensing process after preparation completed, but also the purity of functional ingredients will be insufficient since the solvent remains in the separated or purified products.

### SUMMARY OF THE INVENTION

The objective of the present invention is to provide a method using supercritical fluid technology, which can separate and purify active ingredients from the placenta, featured by low cost, simple manufacturing, good operability, thus is applicable for large-scale production and able to keep the activity of the bioactive ingredients in placenta. Complicated solvent removal, concentrated or separation steps are not required. Supercritical fluid can be recycled and reused without concentrated solvent removal process, solvent residue or other safety concerns. Thus this method is environmental protection, safe and practical.

Accordingly, to achieve the above-mentioned objective, the present invention provides a method to separate and purify the functional ingredients in placenta using supercritical fluid technology, which includes the following steps at least: extraction, i.e. placenta extract as well as supercritical CO₂ solvent is guided into a fractionation tank constantly in a preset velocity at a preset temperature and pressure to extract peptides, proteins and other functional ingredients; purification, i.e. fractionated extract and supercritical CO₂ solvent is then guided into separation tanks at a preset temperature and a pressure lower than said pressure to separate and purify peptides, proteins, active growth factors and other functional ingredients.

### BRIEF DESCRIPTION OF THE DRAWINGS

FIG. 1 is a flow chart of a preferred embodiment of the present invention.

FIG. 2 is the analysis chart of a preferred embodiment about efficiency of peptide separated from placenta.

FIG. 3 is the analysis chart of a preferred embodiment about efficiency of protein separated from placenta

FIG. 4 is the analysis chart of a preferred embodiment about selectivity of peptides/proteins.

FIGS. 5 and 6 are the concentration distribution charts of placenta and protein in placenta extract (Feed), raffinates at the bottom of fractionation tank (R) and in the three separation tanks F1, F2, F3.

FIG. 7 is the analysis chart of a preferred embodiment about ADSC viability.

FIG. 8 is the concentration distribution chart of ADSC viability in placenta extract (Feed), raffinate at the bottom of fractionation tank (R) and in the three separation tanks F1, F2, F3.

### DETAILED DESCRIPTION OF THE INVENTION

Hereby, a preferred embodiment is listed for detailed descriptions in combination with the charts as follows:

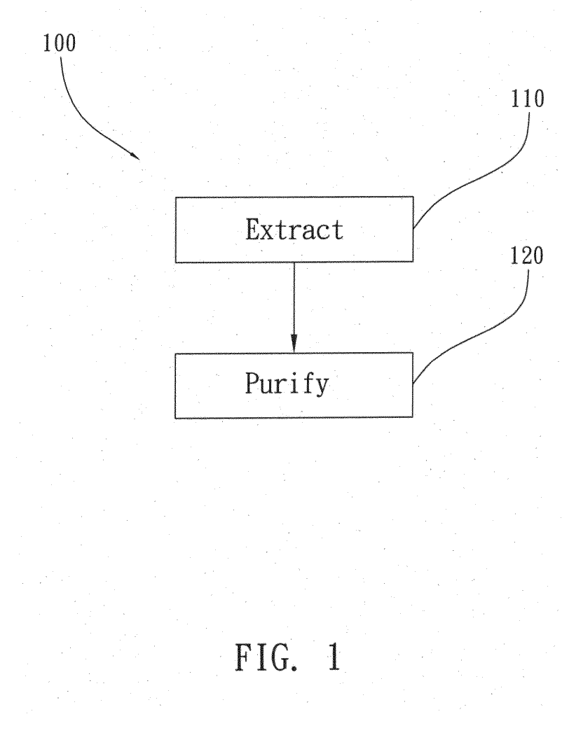

Referring to FIG. 1, a preferred embodiment of a method 100 which separates and purifies the functional ingredients, i.e. peptides, proteins and active growth factors from the placenta using supercritical fluid technology. The first step is to extract 110: 1-2 kg placenta powder (dry placenta powder of human, sheep, pig, deer or other animals) is mixed with ethanol with a proportion of 1:10 (W/V) and centrifuged to prepare placenta extract. The extract is then guided into a fractionation tank consecutively under the operation conditions of pressure 2000-4000 psi, temperature 40-60° C., flow rate of supercritical CO₂ solvent at 3-9 L/hr and flow rate of placenta extract at 1-3 L/hr, so as to extract peptides, proteins and other functional ingredients at the top and bottom of the fractionation tank. The fractionation tank is stainless steel with an inner diameter of 36 mm and a height of 1000 mm, filled with stainless steel monomer piece.

The second step of the present invention is to purify 120: under a temperature of 40° C. or 60° C., peptides, proteins and other functional ingredients extracted from the fractionation tank is conveyed through the three separation tanks consecutively and depressurized to 3000, 2000, 1000 psi respectively, i.e. the pressure in the first tank is 3000 psi, that of the second tank is 2000 psi and the pressure in the third tank is 1000 psi, so as to separate supercritical CO₂ solvent and the peptides, proteins and other functional ingredients. The separation tanks are all stainless steel with an inner diameter of 36 mm and a height of 500 mm.

Said method is carried out under different operation conditions (varied pressures and temperatures), i.e. placenta extract (Feed), extract on the top of fractionation tank, raffinate (R) at the bottom of fractionation tank and functional ingredients such as peptides, proteins and active growth factors obtained from the three separation tanks (named as differentiated objects F1, F2, F3) are taken to analyze the following biochemical properties: (1) viability of adipose derived stem cells (ADSC); (2) protein concentration; (3) protein electrophoresis; (4) peptide concentration.

Viability of ADSC, represented by %, is measured by flow cytometer. Protein concentration, represented by mg/g dw, is determined quantitatively by the Bradford reagent test. Protein is analyzed by SDS-PAGE protein gel electrophoresis, quantified and represented by mg/g dw. Peptide concentration, represented by mg/g dw, is analyzed quantitatively by HPLC analysis.

Experimental results can be divided into two parts (1) functional ingredients in placenta are optimally applicable for separation and purification conditions; (2) optimal production process.

Firstly, the peptides, proteins, active growth factors and other functional ingredients in placenta extract are extracted and separated in the continuous fractionation system using supercritical fluid technology. The top and bottom of the fractionation samples are collected, analyzed and calculated the separation efficiencies, as the K-value and selectivity, in a fractionation system. Kpeptides and Kproteins are defined as the peptide and protein concentration on the top part divided by the peptide and protein concentration at the bottom part, respectively. The selectivity of peptides/proteins is defined the Kpeptides value divided by the Kproteins value.

Kpeptides=1 indicates the peptide concentration on the top part equal to the peptide concentration at the bottom part; Kpeptides>>1 indicates that the higher concentration of peptide will be extracted on the top of fractionation tank; Kproteins<<1 indicates that the higher concentration of protein will be extracted at the bottom of fractionation tank.

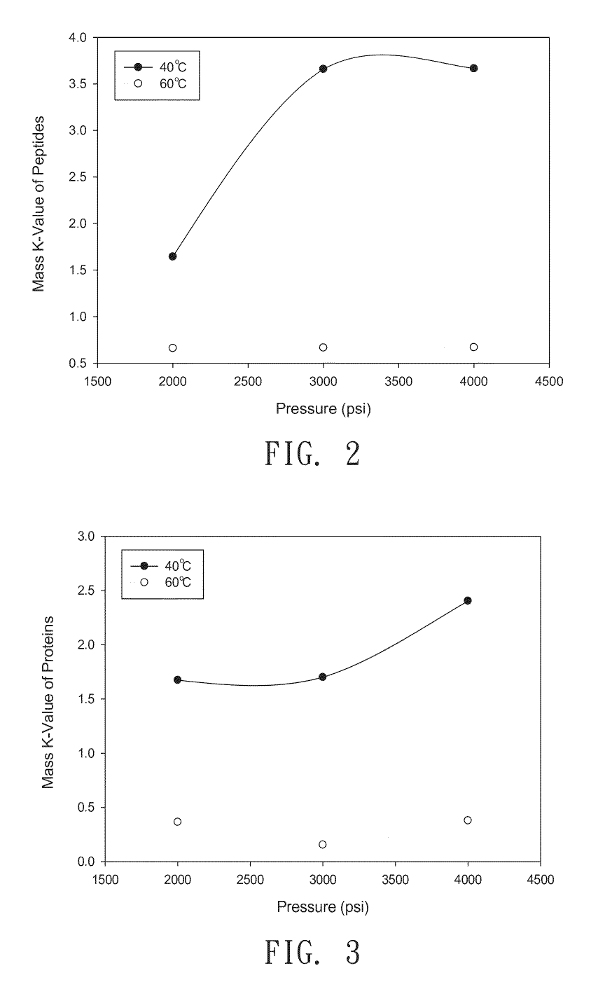

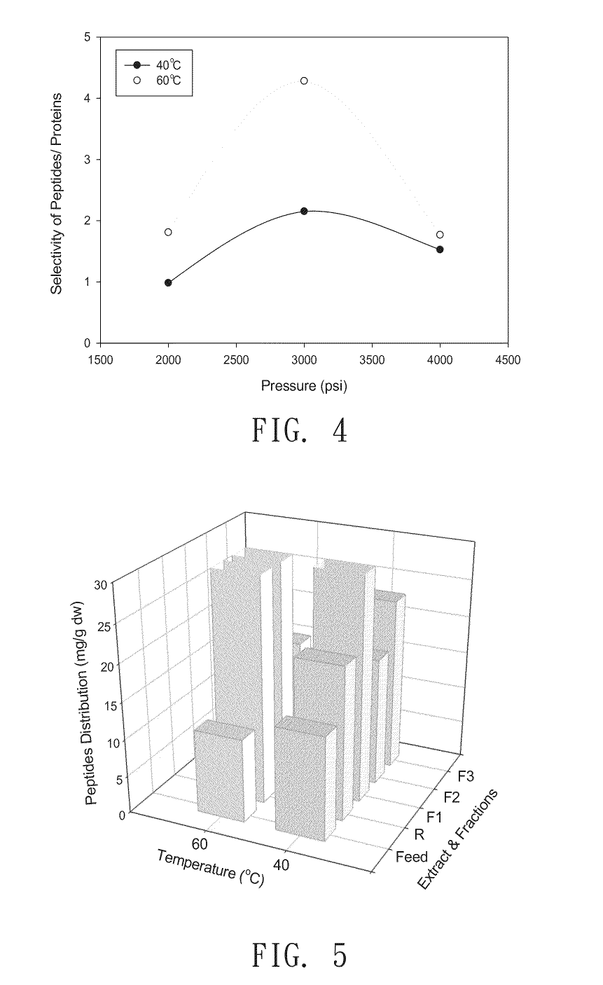

The separation efficiencies of placenta peptide and protein are dependent on the operating pressure and temperature using supercritical fluid technology. Under certain ranges of temperature and pressure, supercritical CO₂ became a solvent to extract the active ingredients. Indicated by the values after fractionation, in FIG. 2, at a temperature of 40° C. and a pressure higher than 3000 psi, the separation efficiency of placenta peptide (Kpeptides) is up to 3.0; It indicates that the higher concentration of peptides will be extracted on the top of fractionation tank. The separation efficiency of placenta protein (Kproteins) is up to 2.5, as shown in FIG. 3, at a temperature of 40° C. and a pressure higher than 4000 psi; It indicates that the higher concentration of proteins will be extracted on the top of fractionation tank. Therefore, temperature 40° C. and pressure 4000 psi are the optimal condition for extracting placenta peptides and proteins on top of fractionation. The selectivity of fractionation with supercritical CO₂ is based on CO₂ density performed by changing pressure and/or temperature. The selectivity of peptides/proteins is up to 4.0, as shown in FIG. 4, at a temperature of 60° C. and a pressure 3000 psi. Therefore, temperature of 60° C. and pressure 3000 psi are the optimal condition for separating placenta peptides and proteins into the three separation tanks (F1, F2, F3).

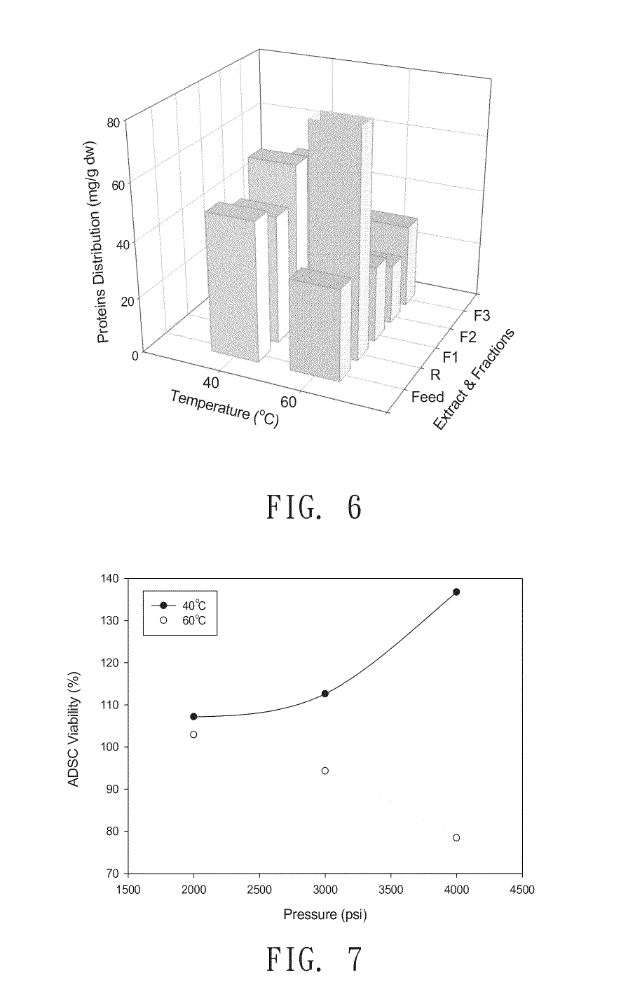

For example, placenta peptides and proteins are first extracted on the top of fractionation tank under the operation temperature 40° C. and pressure 4000 psi. It is then subjective to successive partitioning using supercritical CO₂ at temperature 60° C. and pressures of 3000, 2000, 1000 psi to yield F1, F2 and F3 fractions, respectively. FIG. 5 and FIG. 6 shows the concentration distribution of peptides and proteins in placenta extract (Feed), raffinate at the bottom of fractionation tank (R) and in the triple-separation tank F1, F2, F3, respectively. Thus, under the operation conditions of temperature 40° C. and pressure 4000 psi, most of the placenta protein is separated at the bottom of fractionation tank. The highest concentrations of the placenta peptides remain in the F1 and F2 separation tanks under the operation conditions of temperature 60° C. and pressures 3000 or 2000 psi.

Indicated by the experimental results in FIG. 7, the highest ADSC viability of collected samples is under the operation pressure 4000 psi and temperature 40° C. This optimal condition is easily to extract functional ingredients to activate ADSC growth factor, so as to promote the activation and generation ability of adipose tissue.

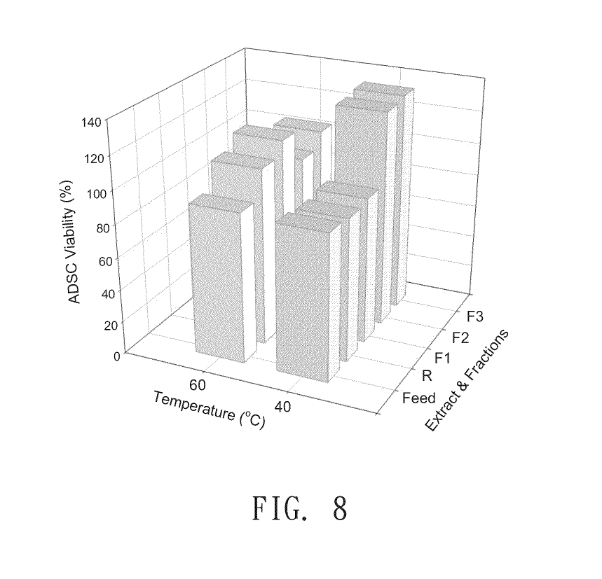

FIG. 8 shows the distribution of ADSC viability in placenta extract (Feed), raffinate (R) and fractions F1, F2, F3, respectively. The optimal condition for ADSC viability is experimentally analyzed in the second (fractions F2) and third (fractions F3) separation tanks under the operation conditions of temperature 40° C. and pressures 2000 or 1000 psi.

Moreover, to validate the effect of the present invention applied in functional food and cosmetic industries, the following industrial mass production tests are carried out:

Supercritical fluid are extracted together with the placenta extract at a temperature of 40° C. and pressure of 4000 psi. At the same time, growth factor most applicable to promote the activation and regeneration abilities of adipose derived stem cells (ADSC) in placenta is separated and activated at a temperature of 40° C. and pressure of 4000 psi in the second separation slot, i.e. fractionation matter F2. Data analysis about experiment formulas and results are shown in Table 1. Functional ingredients such as peptides, proteins and activation factors separated and purified from placenta through the present invention will promote the growth factor for ADSC activation and regeneration effectively when added in functional beverages, food (jelly) or essence and promote the activation ability of ADSC by 15-21%.

TABLE 1

| | | ADSC |
|---|---|---|
| | | viability |
| Item | Main components | (%) |
| Placenta jelly | Placenta fraction (F1, F2, F3), collagen, | 121 |
| | fragrance | |
| Placenta beverage | Placenta fraction (F1, F2, F3), sugar, | 115 |
| | fragrance | |
| Placenta essence | Placenta fraction (F1, F2, F3), collagen, | 118 |
| | hyaluronic acid, fragrance | |

From the foregoing, the present invention provides a method to separate and purify functional ingredients in placenta through supercritical fluid technology, which applies safe and nontoxic supercritical solvent, in combination with physical extraction and purification steps to separate the activation ingredients, such as peptide and protein from placenta extract. There is no safety concern such as solvent residue, thus is environmental protection and safe and the supercritical solvent can be recycled and reused. Accordingly, the present invention is applicable for mass production and continuous operation compared to the existing extraction and separation methods of placenta peptide and protein, with significant practical value.

1. A method which separates and purifies functional ingredients in placenta using supercritical fluid technology comprises the following steps:
extraction: placenta extract as well as supercritical CO₂ solvent, is guided into a fractionation tank constantly in a preset velocity at a preset temperature and pressure to extract peptides, proteins and other functional ingredients;
purification: fractionated extract and supercritical CO₂ solvent is then guided into separation tanks at a preset temperature and a pressure lower than said pressure to separate and purify peptides, proteins, active growth factors and other functional ingredients.

2. The method defined in claim 1, wherein in the purification step, functional ingredients extracted from the fractionation tank is conveyed through the three separation tanks consecutively and depressurized gradually, so as to separate, fractionate and purify the content of placenta peptide and protein.

3. The method defined in claim 1, wherein in the extraction step, the placenta extract is mixed and centrifuged by placenta powder and ethanol in a proportion of 1:10 (W/V).

4. The method defined in claim 3, wherein placenta powder is dried powder of human, sheep, pig, deer or other animals' placenta.

5. The method defined in claim 1, wherein in the extraction step, namely placenta extract is placed in fractionation tank at a pressure of 2000-4000 psi and temperature of 40° C. or 60° C.; supercritical CO₂ solvent into the fractionation tank under the conditions of flow rate of supercritical fluid at 3-9 L/hr and flow rate of placenta extract at 1-3 L/hr.

6. The method defined in claim 2, wherein in the purification step, three separation tanks are depressurized rapidly to a pressure of 3000, 2000, 1000 psi at the temperature of 40° C. or 60° C., so as to separate the supercritical CO₂ solvent.

7. The method defined in claim 5, wherein in the extraction step, the most applicable operation condition is: pressure 4000 psi and temperature 40° C.

8. The method defined in claim 6, wherein in the purification step, the most applicable condition is: placenta peptide and protein are mostly applicable to be purified and fractionated into the three separation tanks at a temperature of 60° C. and a pressure of 3000 psi.

9. The method defined in claim 6, wherein in the purification step, ADSC viability is mostly applicable to be purified and fractionated in the second and third separation tanks under the operation conditions of temperature 40° C. and pressures 2000 or 1000 psi.
